(19) United States
(12) Patent Application Publication
(10) Pub. No.: US 2010/0137613 A1
(43) Pub. Date: Jun. 3, 2010

(54) PROCESS FOR EPROSARTAN

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Medabalimi Peter Paul Raj, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 11/995,101

(22) PCT Filed: Dec. 27, 2006
(86) PCT No.: PCT/IN06/00507

§ 371 Date: Jan. 8, 2008

(51) Int. Cl. C07D 233/64

(52) U.S. Cl. 548/315.1

(57) ABSTRACT

The present invention provides an improved and commercially viable process for preparation of eprosartan and its pharmaceutically acceptable acid addition salts thereof in high purity and in high yield. Thus, for example, methyl 4-[[2-butyl-5-formyl-1 H-imidazol-1-yl]methyl]benzoate is reacted with ethyl 2-carboxy-3-(2-thienyl)propionate in the presence of a base, such as piperidine or piperidinium propionate in propionic acid, in cyclohexane solvent to give ethyl (αE)-α-[(2-n-butyl-1-[(4-(methoxy-carbonyl) phenyl]methyl]-1 H-imidazol-5-yl]methylene-2-thiophene propionate substantially free of decarboxylate impurity namely, ethyl 3-(2-thienyl)propionate, which is then subjected to base hydrolysis followed by treatment with methanesulfonic acid to obtain eprosartan mesylate in high purity and in high yield.

### FIELD OF THE INVENTION

The present invention provides an improved and commercially viable process for preparation of eprosartan and its pharmaceutically acceptable acid addition salts thereof in high purity and in high yield.

### BACKGROUND OF THE INVENTION

U.S. Pat. No. 5,185,351 disclosed a variety of imidazolylalkenoic acid derivatives, processes for their preparation, pharmaceutical compositions in which they are present and use thereof. These compounds are angiotensin II receptor antagonists and are useful in regulating hypertension induced or exacerbated by angiotensin II, and in the treatment of congestive heart failure, renal failure, and glaucoma. Among them, eprosartan mesylate, chemically (αE)-α-[[2-n-Butyl-1-[(4-carboxyphenyl)methyl]-1H-imidazol-5-yl]methylene-2-thiophenepropanoic acid monomethanesulfonate is a promising angiotensin II receptor antagonist useful in the treatment of hypertension, congestive heart failure and renal failure. Eprosartan is represented by the following structure:

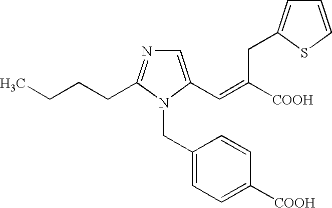

Processes for the preparations of eprosartan and related compounds were disclosed in U.S. Pat. No. 5,185,351, PCT publication No. 98/35963 A1 and European Patent No. 0973769 B1.

According to the U.S. Pat. No. 5,185,351 (herein after referred to as '351 patent), methyl 4-[2-butyl-5-formyl-1H-imidazol-1-yl]methyl]benzoate is reacted with ethyl 2-carboxy-3-(2-thienyl)propionate, in the presence of a base, such as piperidine, in a suitable solvent, such as toluene, at a temperature of 80° C. to 110° C., preferably at 100° C., to give ethyl (αE)-α-[[2-n-Butyl-1-[[4-(methoxycarbonyl)phenyl]methyl]-1H-imidazol-5-yl]methylene-2-thiophene propionate, which is then hydrolyzed with a base such as sodium hydroxide to give eprosartan, which is further converted to eprosartan mesylate.

We have repeated the eprosartan synthetic procedure described in the '351 patent and found that relatively large amounts of impurities were obtained along with ethyl (αE)-α-[[2-n-Butyl-1-[[4-(methoxycarbonyl)phenyl]methyl]-1H-imidazol-5-yl]methylene-2-thiophene propionate when toluene is used as the solvent in the reaction between methyl 4-[[2-butyl-5-formyl-1H-imidazol-1-yl]methyl]benzoate and ethyl 2-carboxy-3-(2-thienyl)propionate in presence of piperidine at reflux temperature (100-120° C.), and hence the yield of the product is very poor (6-7%). If the above reaction is carried out in toluene without refluxing at 80-90° C. the reaction is not going forward.

In a specific run, we have found that ethyl (αE)-α-[[2-n-Butyl-1-[[4-(methoxycarbonyl)phenygmethyl]-1H-imidazol-5-yl]methylene-2-thiophene propionate prepared by the above procedure, contained 65-70% of the decarboxylate impurity namely, ethyl 3-(2-thienyl)propionate, and 23-30% of some other impurities. It is observed that the decarboxylate impurity is further carried over to the next step, which is also converted to 3-(2-thienyl)propanoic acid during hydrolysis reaction with sodium hydroxide and found that it appeared as an impurity in eprosartan. The process described in the '351 patent also involves column chromatographic purifications.

Based on the aforementioned drawbacks, this process finds to be unsuitable for preparation of eprosartan at lab scale and commercial scale operations.

We have found that the formation of large amounts of the decarboxylate impurity in the above reaction is due to the degradation of 2-carboxy-3-(2-thienyl)propionate.

The '351 patent further described another process for preparation of eprosartan by using lithium derivatives such as n-butyl lithium. This process also suffers from drawbacks since it would be very difficult to handle lithium derivatives in large-scale scale operations, thereby making the process commercially not viable.

According to U.S. Pat. No. 6,172,237 B1, eprosartan is prepared by reacting 4-[[2-butyl-5-formyl-1H-imidazol-1-yl]methyl]benzoic acid or the bisulfite addition compound of 4-[[2-butyl-5-formyl-1H-imidazol-1-yl]methyl]benzoic acid with (2-thienylmethyl)-propanedioic acid, mono-ethyl ester in a solvent (and/or solvent systems) selected from the group consisting of toluene, cyclohexane, cyclohexane:dichloroethane (12:5 or 1:1), cyclohexane:pyridine (12:5), and cyclohexane:ethyl acetate:pyridine (8:3:1) in the presence of piperidine as catalyst at reflux temperature at reduced pressure followed by hydrolysis of the intermediate ethyl ester (ethyl (αE)-α-[[2-n-butyl-1-[[4-(methoxycarbonyl) phenyl]methyl]-1H-imidazol-5-yl]methylene-2-thiophene propionate).

The yields of eprosartan obtained according to the processes described in the U.S. Pat. No. 6,172,237 B1 are very low, this is due to the yield loss resulted during the hydrolysis of methyl 4-[[2-butyl-5-formyl-1H-imidazol-1-yl]methyl]-benzoate to obtain 4-[[2-butyl-5-formyl-1H-imidazol-1-yl]methyl]benzoic acid. Moreover, it is difficult to maintain the reaction at reflux under vacuum. The vacuum creates loss of solvent from reaction medium. So there is a need to add extra solvent to the reaction medium.

European Patent No. 0973769 provides processes for the preparation of eprosartan by using specific regioselective nitrogen-protecting reagents such as C₁₋₄-alkyl ester derivatives of acrylic acid.

The preparation of eprosartan as described in the European Patent No. 0973769 involves a lengthy process, the yields obtained in this process are very low and also the process is not satisfactory from purity point of view.

However, a need still remains for an improved and commercially viable process of preparing pure eprosartan that solving the aforesaid problems associated with processes described in the prior art, which will be suitable for large-scale preparation, in terms of simplicity, chemical yield and purity of the product.

We have found that the formation of the decarboxylate impurity in the preparation of ethyl (αE)-α-[[2-n-butyl-1-[[4-(methoxycarbonyl) phenyl]methyl]-1H-imidazol-5-yl]methylene-2-thiophene propionate can be reduced or avoided with the use of cyclohexane or n-hexane as solvent to obtain eprosartan in high purity and in high yield.

According to the novel process, no chromatographic separations are required for isolating pure eprosartan there by making the process commercially viable.

### DETAILED DESCRIPTION OF THE INVENTION

In accordance with the present invention, there is provided a process for preparing eprosartan of formula I:

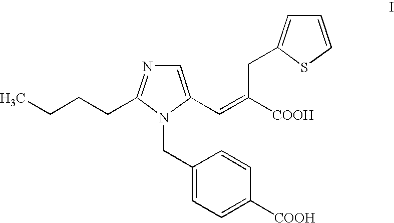

or a pharmaceutically acceptable salt thereof; which comprises:

a) reacting methyl 4-[[2-butyl-5-formyl-1H-imidazol-1-yl]methyl]benzoate of formula II:

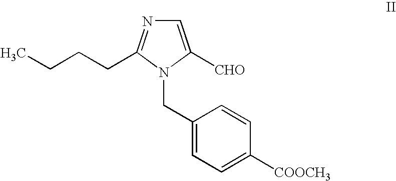

with ethyl 2-carboxy-3-(2-thienyl)propionate of formula III:

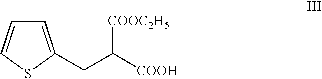

in the presence of a base in a solvent selected from cyclohexane and n-hexane, to give diester intermediate of formula IV:

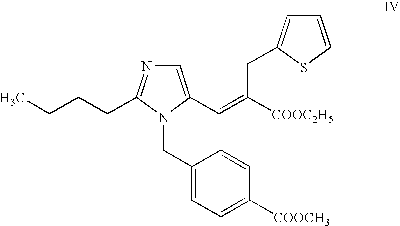

substantially free of decarboxylate impurity namely, ethyl 3-(2-thienyl)propionate; and b) hydrolyzing the compound of formula IV with a base such as sodium or potassium hydroxide to obtain pure eprosartan of formula I and optionally converting eprosartan formed into a pharmaceutically acceptable acid addition salts of eprosartan.

The term “diester intermediate substantially free of decarboxylate impurity” refers to the diester intermediate containing the content of decarboxylate impurity in less than about 35% by weight, preferably less than about 10% by weight and still more preferably less than about 5% by weight of diester intermediate.

The reaction in step-(a) may be carried out between 60° C. and reflux temperature of the solvent used, preferably carried out between 65° C. and reflux temperature of the solvent used, and still more preferably carried out at reflux temperature of the solvent used. Preferable solvent used in the reaction in step-(a) is cyclohexane.

Preferable base used in the reaction in step-(a) is selected from the group comprising piperidine, morpholine, 1-methylpiperazine, pyrrolidine and a salt thereof. More preferable base is piperidine or piperidinium propionate.

The reaction mass containing the diester intermediate of formula IV obtained in step-(a) may be subjected to usual work up. The reaction mass may be used directly in the next step to produce eprosartan or its pharmaceutically acceptable acid addition salts, or the diester intermediate of formula IV may be isolated and then used in the next step.

After completion of the hydrolysis reaction in step-(b), the reaction mass may then be treated with hydrochloric acid followed by usual work up such as washings, extractions etc.

The novel process provides eprosartan in high yield and purity, thus obviating the need to use column chromatography to purify the material.

The hydrolysis reaction in step-(b) may be carried out by known methods for example as described in the U.S. Pat. No. 5,185,351.

Pharmaceutically acceptable acid addition salts of compounds of eprosartan are formed with appropriate organic or inorganic acids by methods known in the art.

Preferable pharmaceutically acceptable. acid addition salts of eprosartan, but not limited to, are obtained from hydrochloric acid, hydrobromic acid, hydroiodic acid, methanesulfonic acid, benzenesulfonic acid, maleic acid, fumaric acid, benzoic acid, ascorbic acid, succinic acid, and more preferable salt being eprosartan mesylate.

The following examples are given for the purpose of illustrating the present invention and should not be considered as limitation on the scope or spirit of the invention.

### REFERENCE EXAMPLES

### Reference Example 1

Step-(a):

Potassium carbonate (246.3 gm) and dimethylformamide (960 ml) are added to 2-butyl-4-chloro-1H-imidazole-5-carbaldehyde (245 gm), the contents are stirred for 30 minutes at 25-30° C. and then methyl 4-(bromomethyl)benzoate (334 gm) is added to the reaction mass at once at 25-30° C. The reaction mass is heated to 70° C., stirred for 12 hours and then cooled to 20-25° C. Filtered the mass, washed the solid with ethyl acetate (668 ml), to the resulting filtrate added ethyl acetate (668 ml) followed by 10% NaCl solution (260 gm of NaCl in 2627 ml of water) and then stirred for 30 minutes. Separated the layers and collected the ethyl acetate layer. To the aqueous layer added ethyl acetate (668 ml), stirred for 30 minutes, separated the layers, combined the total ethyl acetate layer and then stirred with 10% NaCl solution (69 gm of NaCl in 695 ml of water) for 30 minutes. Separated the layers, combined the total ethyl acetate layer and passed through sodium sulphate (10 gm). The ethyl acetate layer is distilled under vacuum at below 50° C. and co-distilled with isopropyl alcohol (348 ml). To the resulting mass added isopropyl alcohol (1667 ml) and charcoal (11.7 gm) and then the contents are refluxed for 1 hour. The reaction mass is filtered through hyflow bed, washed the bed with hot isopropyl alcohol (478 ml), the resulting filtrate is initially cooled to 25-30° C. and latter cooled to 0-5° C. The reaction mass is stirred for 2 hours, filtered the mass, washed with chilled isopropyl alcohol (145 ml), suck dried the material and then dried at 70° C. to give 265 gm of methyl 4-[[2-butyl-4-chloro-5-formyl-1H-imidazol-1-yl]methyl]benzoate.

Step-(b):

The mixture of methyl 4-[[2-butyl-4-chloro-5-formyl-1H-imidazol-1-yl]methyl]benzoate (214 gm), 5% Pd/C (143.3 _{g}m), potassium acetate (70.7 gm) and methanol (2292 ml) is taken in a hydrogenation flask and then subjected to hydrogenation under hydrogen pressure of 12 Kg for 8 hours at 25-30° C. The reaction mass is filtered through hyflow bed, washed the bed with methanol (1900 ml) and the resulting filtrate is distilled under vacuum at below 50° C. To the residue added ethyl acetate (2568 ml) and water (2782 ml) at 25-30° C., adjusted the resulting mass pH to 8-9 with 5% sodium carbonate solution at 25-30° C. (64.2 gm Na₂CO₃ in 1284 ml of water) and then separated the layers. The organic layer is washed with brine solution (21.4 gm of NaCl in 2782 ml of water), separated the layers and the resulting organic layer is passed through sodium sulphate (10 gm). The organic layer is distilled under vacuum at below 50° C., activated MnO₂ (80 gm) and chloroform (856 ml) are added to residue and then refluxed for 4 hours. The resulting mass is filtered through hyflow bed and washed the bed with chloroform (382 ml). To the filtrate added charcoal (10 gm), refluxed for 30 minutes, filtered the mass and washed the bed with chloroform (250 ml). The filtrate is distilled under vacuum to form an oily mass and then cooled until to form a solid to give 168 gm of methyl 4-[[2-butyl-5-formyl-1H-imidazol -1-yl]methyl]benzoate.

### Reference Example 2

Step-(a):

Piperidine (31.6 gm), benzoic acid (0.448 gm), diethyl malonate (312 gm) and cyclohexane (1240 ml) are added to 2-thiophenecarboxaldehyde (200 gm) under stirring at 25-30° C., the contents are refluxed under dean stark for 20 hours and separated the water generated from the reaction mass. The reaction mass is distilled under vacuum, to the residue added toluene (1000 ml) followed by addition of 10% HCl solution (3×160 ml) and then stirred for 30 minutes. The resulting organic layer is washed with saturated sodium bicarbonate solution (3×160 ml) followed by brine solution (20 gm of NaCl in 200 ml of water) and then stirred with charcoal (8 gm) for 30 minutes at 50-60° C. Filtered the mass on hyflow bed, washed the bed with hot toluene (50 ml) and the resulting filtrate is concentrated to give 466 gm of 2-thienylidene malonate as residue.

Step-(b):

Ethanol (2340 ml) is added to 2-thienylidene malonate (466 gm, obtained in step-a) under stirring at 25-30° C., the contents are cooled to 0-5° C. and then sodium borohydride (42.8 gm) is slowly added during 2 hours at 0-5° C. The contents are stirred for 4 hours at 0-5° C., raised the mass temperature to 25-30° C. and then adjusted the pH to 6 with acetic acid (255 ml) at 25-30° C. Filtered the mass, washed with ethanol (100 ml) and distilled the filtrate under vacuum at below 50° C. To the residue added toluene (1876 ml) and water (1410 ml), stirred for 30 minutes and separated the layers. The organic layer is washed with water (940 ml) followed by brine solution (15 gm of NaCl in 150 ml of water) and the resulting organic layer is then subjected to carbon treatment. Filtered the mass through hyflow bed, washed the bed with hot toluene (100 ml) and the resulting filtrate is concentrated to give 355 gm of diethyl (2-thienylmethyl)malonate as residue.

Step-(c):

Diethyl (2-thienylmethyl)malonate (355 gm, obtained in step-b) is added to ethanol (1037 ml) under stirring at 25-30° C., to the reaction mass added KOH solution (76.6 gm of KOH in 11.25 ml of water and 2071 ml of ethanol) drop wise during 2-3 hours by maintaining the temperature between 25-35° C. The reaction mass is stirred for 48 hours at 25-35° C. and then distilled the mass under vacuum at below 50° C. To the residue added water (1043 ml) and toluene (1043 ml), stirred for 30 minutes, separated the layers and discarded the toluene layer. The aqueous layer pH is adjusted to 1 with 2N H₂SO₄ solution drop wise (65.7 nil of H₂SO₄ in 531 ml of water), toluene (2×1043 ml) is added to the resulting mass, stirred for 30 minutes and then separated the layers. Combined both the organic layers, washed with water (424 ml) followed by 10% NaCl solution (42 gm of NaCl in 420 ml of water) and the resulting organic layer is then subjected to carbon treatment. Filtered the mass through hyflow bed, washed with toluene (104 ml) and the resulting filtrate is distilled under vacuum until completely removed the traces of toluene to give 248 gm of ethyl 2-carboxy-3-(2-thienyl)propionate.

### EXAMPLES

### Example 1

Methyl 4-[[2-butyl-5-formyl-1H-imidazol-1-yl]methyl]benzoate (32 gm) and ethyl 2-carboxy-3-(2-thienyl)propionate (57.15 gm) are added to cyclohexane (292 ml) under stirring at 25-30° C., the contents are heated to reflux (80-85° C.) for 2 hours under dean stark to separate the traces of water. The reaction mass is cooled to 50° C. and then slowly added a freshly prepared catalyst solution of propanoic acid (22.93 ml) in cyclohexane (53 ml) and piperidine (10.66 ml). The resulting mass is heated to reflux (80-85° C.) for 20 hours, to the reaction mass drop wise added 50% NaOH solution (64 gm of NaOH in 256 ml of water) after reflux at 50° C. and then the reaction mass is heated to reflux for 2 hours. The reaction mass is cooled to 60° C., separated the layers, to the aqueous layer added ethanol (192 ml) and then pH of the mass is adjusted to 5.0 to 5.1 at 60° C. with 6N HCl solution (66 ml of HCl and 66 ml of water). The resulting mass cooled to 20-25° C. and stirred for 2 hours. Filtered the mass, washed with water (100 ml) and then dried at 70-75° C. to give 135 gm of (αE)-α-[[2-n-Butyl-1-[(4-carboxyphenyl)methyl]-1H-imidazol-5-yl]methylene -2-thiophenepropanoic acid (eprosartan base, HPLC purity: 98.2%).

### Example 2

Methyl 4-[[2-butyl-5-formyl-1H-imidazol-1-yl]methyl]benzoate (15 gm) and ethyl 2-carboxy-3-(2-thienyl)propionate (27 gm) are added to cyclohexane (138 ml) under stirring at 25-30° C., the contents are heated to reflux for 2 hours under dean stark to separate the traces of water. The reaction mass is cooled to 50° C. and then slowly drop wise added a freshly prepared catalyst solution of propanoic acid (10.8 ml) in cyclohexane (25 ml) and piperidine (5 ml). The resulting mass is heated to reflux for 15 hours, cooled the mass to 25-30° C. and then distilled under vacuum at 50° C. The resulting oily mass is stirred with toluene (60 ml) and water (25 ml), separated the layers and the organic layer is again washed with water (25 ml). Separated the layers, to the organic layer added water (120 ml) and ethanol (150 ml) and then adjusted the pH of the mass to 1 with 15% HCl solution (86 ml). Separated the layers and the aqueous layer pH is adjusted to 6.0 with 10% NaOH solution. The resulting mass is extracted with toluene (2×50 ml), separated the layers and collected the organic layer. Combined both the organic layers and then distilled under vacuum to give 16 gm of ethyl (αE)-α-[[2-n-butyl-1-[[4-(methoxycarbonyl)phenyl] methyl]-1H-imidazol-5-yl]methylene-2-thiophene propionate (HPLC purity: 90%).

### Example 3

Acetic acid (474 ml) is added to eprosartan free base crude (158 gm, obtained in example 1) under stirring at 25-30° C., the contents are heated to 80° C. until to form a clear solution and then stirred with charcoal (2 gm) at 80° C. for 30 minutes. Filtered the mass through hyflow bed, washed the bed with hot acetic acid (158 ml), the resulting filtrate is cooled to 25-30° C. and then stirred for 1 hour. To the reaction mass added ethyl acetate (1580 ml) and stirred for 2 hours. Filtered the solid, washed with ethyl acetate (376 ml) and then dried at 40° C. under vacuum to give 143 gm of pure eprosartan free base (HPLC purity: 99.5%).

### Example 4

Eprosartan free base (135 gm) is stirred with isopropyl alcohol (2000 ml), the reaction mass is cooled to 0-5° C. and then methane sulfonic acid (91.8 gm) is added drop wise to the mass at 0-5° C. The reaction mass is stirred for 5 hours at 0 - 5° C., filtered the mass, washed the material with isopropyl alcohol (375 ml) and then dried under vacuum at 45° C. to give 158 gm of eprosartan mesylate (HPLC purity: 99.9%).

We claim:

1. A process for preparation of eprosartan of formula I: 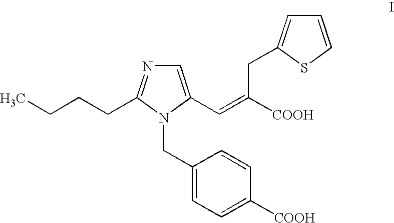
or a pharmaceutically acceptable salt thereof; which comprises:
to a) reacting methyl 4-[[2-butyl-5-formyl-1H-imidazol-1-yl]methyl]benzoate of formula II: 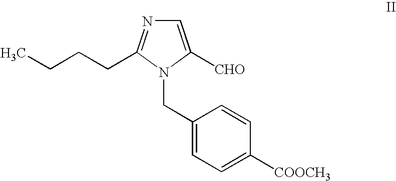
with ethyl 2-carboxy-3-(2-thienyl)propionate of formula III: 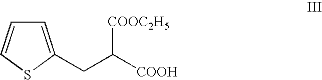
in the presence of a base in a solvent selected from cyclohexane and n-hexane to give diester intermediate of formula IV: 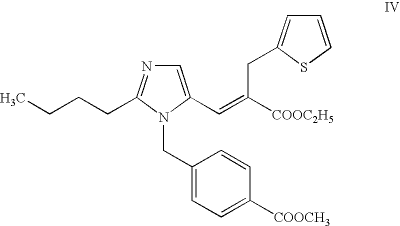
substantially free of decarboxylate impurity namely, ethyl 3-(2-thienyl)propionate; and
b) hydrolyzing the compound of formula IV with a base such as sodium or potassium hydroxide to obtain pure eprosartan of formula I and optionally converting eprosartan formed into a pharmaceutically acceptable acid addition salts of eprosartan.

2. The process as claimed in claim 1, wherein the diester intermediate of formula IV obtained is containing the content of decarboxylate impurity in less than about 35% by weight.

3. The process as claimed in claim 2, wherein the diester intermediate of formula IV containing the content of decarboxylate impurity in less than about 10% by weight.

4. The process as claimed in claim 3, wherein the diester intermediate of formula IV containing the content of decarboxylate impurity in less than about 5% by weight.

5. The process as claimed in claim 1, wherein the reaction in step (a) is carried out between 60° C. and reflux temperature of the solvent used.

6. The process as claimed in claim 5, wherein the reaction is carried out between 65° C. and reflux temperature of the solvent used.

7. The process as claimed in claim 6, wherein the reaction is carried out at reflux temperature of the solvent used.

8. The process as claimed in claim 1, wherein the solvent used in the step (a) is cyclohexane.

9. The process as claimed in claim 1, wherein the base used in the reaction in step-(a) is selected from the group comprising piperidine, morpholine, 1-methylpiperazine, pyrrolidine and a salt thereof.

10. The process as claimed in claim 9, wherein the base is piperidine or piperidinium propionate.

11. A compound of formula IV having the content of decarboxylate impurity in less than about 35% by weight.

12. The compound as claimed in claim 11, wherein the compound of formula IV having the content of decarboxylate impurity in less than about 10% by weight.

13. The compound as claimed in claim 12, wherein the compound of formula IV having the content of decarboxylate impurity in less than about 5% by weight.
